(19) United States
(12) Patent Application Publication
(10) Pub. No.: US 2021/0022648 A1
(43) Pub. Date: Jan. 28, 2021

(54) DNA Medical Identification Bracelet

(71) Applicant: Peter Moalemzadeh, La Mesa, CA (US)

(72) Inventor: Peter Moalemzadeh, La Mesa, CA (US)

(21) Appl. No.: 16/203,095

(22) Filed: Nov. 28, 2018

Related U.S. Application Data
(60) Provisional application No. 62/595,380, filed on Dec. 6, 2017.

(51) Int. Cl. A61B 5/11; A61B 5/00; A61B 5/024; A61B 5/1171; A61B 5/1172; G16H 10/65

(52) U.S. Cl. CPC A61B 5/1112; A61B 5/6824; A61B 5/746; A61B 5/024; A61B 5/747; G16H 10/65; A61B 5/1176; A61B 5/1172; A61B 5/0004; A61B 5/0022; A61B 5/742

(57) ABSTRACT

A wearable, secure medical-record storage and management USB/Micro USB bracelet provided together with systems and methods for inputting, managing and updating records contained in such a device. Additionally provided are GPS system for locating the user, pulse monitor for detecting the users pulse, high impact sensor in case of a car accident, or fall, or in case the user is unconscious, which can alert emergency assistance and relay information in emergency situations. Access to the contents of the medical record storage and management USB/Micro USB bracelet is controlled using encrypted software as well as other means for authentication.

### PURPOSE

Records pertinent medical information that will aid EMS and ER staff in the event of a medical emergency (e.g. natural disaster, pandemic).
Creates faster triage times for ER's to receive and treat the patient.
Stores and organizes charts from doctor's visits through simultaneous streaming of data between the doctor's chart and the DNA database.
Reduces and can potentially eliminate medical malpractice in the field.
Delivers live data to improve field communication and system updates that adapt with changes in the medical field such as in the event of an epidemic or pandemic.
Aids in the safety of EMS and First Responders.
Locates the user through the DNA GPS system. Alerts family members via text and call in an emergency.
Detects user's pulse through the DNA pulse monitor.
Tracks and displays future doctor's appointments and reminds when to take medication through the LCD screen.
Warns about lethal medication through previous prescribed medication stored onto the device.
Alerts EMS/FIRE/POLICE through the panic button on the device.

Medical Emergency
“Most diagnostic decisions come from the history-taking component of the interview. Yet, studies of clinical patient visits reveal that patients are often not provided the opportunity or time to tell their story history . . . ” (2)
This is because information obtained from the patient's “golden hour” is vital in determining proper treatment in the hospital setting, which is why, “pre hospital care providers are considered an integral part in the Chain of Survival and Recovery.” (1)
Recognizing a patient with a certain medical history can aid in faster detection and treatment.
Data from EMS calls can be better streamed to the hospital, better preparing the receiving hospital for the patient.
This e-communication is important to the diagnosis and treatment of the patient.
Researchers found that improving communication between health providers can reduce patient injuries from medical errors by 30 percent.

Communication
“The [research] team found that a highly risky period was when patients are transferred or “handed off” between medical providers . . . When there's a breakdown in communication, it sets the stage for potential errors . . . Those errors can lead up to 1,000 deaths per day and cost trillions of dollars in healthcare costs each year.” (3)

Compatibility and Triage
The fact that DNA's software will be available to use by multiple healthcare systems will allow patient information to seamlessly transfer from the USB to the chart used by the receiving facility.
This will greatly lower triage times and reduce congestion in Emergency Rooms.
“Comprehensive triage is said to take only 2 to 5 minutes, although one study found that this occurred only 22% of the time.” (4)
“In 2006 the Institute of Medicine released a report that approximately 120 million Americans, roughly one in three Americans, sought care in an emergency room each year.” (6)
The usage of DNA could potentially decrease triage and waiting time by seamlessly transferring patient information to the receiving hospital's system within seconds.
DNA's compatibility to be used in private practices allows pertinent information from each visit to be best stored onto the USB as it is being typed into the main system.
By storing this information, it will greatly reduce medical miscommunication and errors which are the 4^{th} leading cause of deaths in the U.S., ahead of diabetes, AIDS, automobile deaths, and pulmonary disease. (5)

Medical Miscommunications and Errors
According to a study from the prestigious Journal of Patient Safety, as many as 440,000 patients a year die from preventable medical errors. (7)
The average patient is treated by 2-3 doctors annually, and each prescribes a variety of different medications.
The lack of communication between these doctors could potentially lead to adverse drug reactions.
These reactions occur because of the fact that these patients receive multiple prescriptions at varying strengths, some of which may counteract each other or cause more severe side effects when combined together. (8)
“The costs of adverse drug reactions to society are more than $136 billion annually greater than the total cost of cardiovascular diabetic care.” (8)
“Adverse drug reactions can cause injuries or death in one of three hospital patients.” (8)
“In 2009, there were nearly 4.6 million drug related visits to U.S. emergency rooms nationwide, with more than half due to adverse reactions to prescription medications, most of which were being taken exactly as prescribed.” (8)
In 2011, 4.2 billion prescriptions were written in total. (5)
The average American is prescribed 13 medications and fills an average of 12 prescriptions each year. (5) A study in 2012 showed that 46% of American adults take prescription drugs. (5)
It is imperative for these medications to be fully documented with the doctor's orders so that other physicians can realize contraindicated medicines and procedures.
With the usage of DNA, all medications are fully documented onto the USB drive
This information ensures that the drug is correctly administered, and facilitates communication between the doctors the patient visits.
DNA will allow each doctor to recognize the exact dosage of the drug and the duration of its administration to prevent a medical error due to miscommunication.

System Updates and Adaptation
The healthcare industry is continuously growing.
Flexible adaptation to innovations in healthcare makes DNA's software unique
The history taking component in DNA's database has the ability to change as standard procedures evolve in the charts used by hospitals.
One example of this is the Ebola virus epidemic in late 2014. Charting systems across the country updated their history taking component to include a new question: “Have you traveled in the past 21 days?”

EMS/First Responder Safety
According to OSHA, the Occupational Safety and Health Administration, EMTs are exposed to occupational hazards on a daily basis.
“They need to make informed decisions quickly based on initial information that may be incomplete . . . [this is one of] . . . the challenges of treating patients while ensuring personal safety and health.” (9)
DNA eliminates hazards based on unknown information by uncovering medical history that an unresponsive patient is unable to share with first responders during critical treatment en route to the hospital.
“Twenty two percent of all the paramedics surveyed had at least one exposure to blood in the previous year.” (10)
Paramedics are only a small portion of the Emergency Medical Services.
These exposures may have been prevented if the paramedics were aware of the bloodborne pathogens hosted by their patients.

Overview
DNA can potentially save the lives of millions in the United States.
The number of people being harmed by medical miscommunications and errors is constantly increasing.
Through the usage of DNA, a patient's medical records can be safely stored onto a USB
With roughly half of the nation taking prescription drugs, DNA can prevent doctors from prescribing the wrong drug.
DNA's software can grow and evolve as the medical field evolves, making it unique from any other medical storage device.
DNA can potentially save the lives of EMS and first responders as well as the patients subscribed to DNA.

### SOURCES

1. Foundation of Education and Research in Neurological Emergencies http://www.ferne.org/Lectures/prehospital%200501.htm
2. Institute for Healthcare Communication http://healthcarecomm.org/about--us/impact--of-communication--in--healthcare/3.
3. State of Health Blog KQEDNewshttp://blogs.kqed.org/stateotealth/2014/11/25/miscommunication--a--major--cause--of--medical--error--study--shows/4.
4. Chapter 155: Triage http://www.us.elsevierhealth.com/media/us/samplechapters/9781416000877/Chapter%20155.pdf
5. Top Master's in Healthcare Administration http://www.topmastersinhealthcare.com/drugged--America/6.
6. ABC News Medical Unit http://abcnews.go.com/Health/story?id=5884487
7. Forbes Healthcare http://www.forbes.com/sites/leahbinder/2013/09/23/stunning--news--on--preventable--deaths--in--hospitals/8.
8. Mercola Health http://articles.mercola.com/sites/articles/archive/2011/10/26/prescription--drugs--number--one--cause--preventable--death--in--us.aspx
9. Center for Disease Control http://www.cdc.gov/niosh/docs/wp--solutions/2010-139/pdfs/2010-139.pdf
10. OSHA https://www.osha.gov/Publications/OSHA3370--protecting--EMS--respondersSM.pdf

### DESCRIPTION

A wearable medical record storage in the form of a bracelet that is a Micro USB/USB Device with Wireless Communication Capability, Pulse and GPS Monitoring function, Emergency (slip and fall) Help & Amber Alert Function, Prescription and Doctor Appointments display reminder capability.

The present invention is related to the field of secure information storage and management devices. More particularly, the invention relates to wearable devices for secure storage and management of past and present vital medical data with pulse and GPS monitoring for natural disasters such as earthquakes, hurricanes and tornadoes including the capability to monitor and track soldiers on the battlefield, emergency help or “panic” button and “AMBER” alert assistance with prescription and future doctor appointments reminder.

### BACKGROUND

The growing complexity of medical care and the increasing number of professionals involved presents ever-increasing challenges to the delivery of coherent and consistent care. Medical records are key instruments that assist healthcare providers in delivering coherent and consistent care to patients and can include a wide variety of data, for example, physical examination notes, histories of illness or disease, surgical histories, operation reports, obstetric histories, medication status including dosage and duration history, medical allergies, blood type, family histories, immunization histories, growth and developmental data, and other clinical data, such as laboratory test results and diagnostic data, charts, and images (e.g., x-rays, CT scans, electrocardiograms, echocardiography reports, MRIs with complete radiology reports.)

Access to these and other medical data in a current, comprehensive patient record is important for making accurate medical diagnoses and providing successful patient care. However, most patients do not have access to their complete medical records, and records are frequently maintained in multiple sites by multiple care providers so that no one actually has access to a patient's complete file or even the relevant portions of a patient's file.

Most medical records are stored at individual doctors' offices or clinics, and patients seldom obtain and maintain copies of their own medical records. As is frequently the case, patients may be provided care at numerous independent facilities, for example in a doctor's office, at a clinic, in a hospital, in a rehabilitation facility, in a board-and-care facility, in an assisted living facility, or in a nursing home and ER. Each facility is likely to keep distinct medical records pertinent to a patient's continuity of care. Practitioners may not know of or may not have up-to-date, accurate information about the care provided or recommended by other practitioners and at other facilities. Patients themselves are frequently unable to bridge the information gap between different facilities and providers because patients seldom fully comprehend the nature and details of their care and, as noted above, typically do not maintain their own comprehensive medical histories.

Whenever a health care provider lacks complete, up-to-date, and accurate medical records, patient care may be suboptimal, especially in an emergency situation (such as a natural disaster or pandemic), or if the patient is unconscious. In more serious cases, inappropriate drugs or treatments may be prescribed. Diagnostic tests may be needlessly repeated, increasing costs and risks. Additionally, necessary preventive measures may not be taken because subsequent providers may assume prior caregivers have already addressed the subject.

In order to assemble a complete medical history, a patient or new caregiver must trace prior health care facilities and providers and request the transfer of patient records. In many instances, patient records become part of a paper file but not an electronic file so even if they are present in the office of a provider, a complete record may not be readily accessible or transferable. Some facilities only transfer their own records, so records from other facilities, though part of a patient's original file, may not be transferred to the next care provider. After a certain length of time, original records may be destroyed so that even if a patient goes back to an original care provider, the only place the records may exist is as copies in a later doctor's file. Additionally, if a patient is sent to a specialist, the records, if any, received by the specialist are usually incomplete.

Even more urgent problems relating to medical record storage and management arise in the context of emergency situations. In such situations, healthcare decisions must be made rapidly, and there may be little or no time to access available medical records. When no medical records are available in a timely fashion, emergency health care providers must rely on patients themselves to provide critical care information, such as medication allergies, prior or current illness information, current medication data, etc. In some circumstances, the patient may be unable to provide such information accurately. For example, each year in the U.S., over one million patients arrive at emergency departments unconscious. In cases where a patient in an emergency care situation is unconscious or otherwise incapacitated and unable to provide health information, there may be no access to critical medical data and the provider must rely purely on observation and/or generic protocols to guide emergency health care decisions. Many individuals with serious conditions wear medic alert bracelets, but such devices provide limited information by nature and may be overlooked in a rush to provide care.

A wearable, complete and accurate set of patient medical records, (such as a Continuity of Care Document, CCD, or Continuity of Care Record, CCR), would enable medical care providers and facilities to deliver better and more comprehensive care to individuals, increasing safety and efficiency, reducing errors, and decreasing costs. However, with increased portability, there is an attendant concern regarding security of patient medical information. There is therefore an unmet need for a secure, wearable means for transferring, storing, accessing and updating individual healthcare information.

In addition to the foregoing problems and needs, under various circumstances, including emergency patient care in the field, medical records must be accessible in a secure fashion to third parties such as emergency care providers. Patients may not be able to assist in medical record retrieval under such circumstances, in which case secure access to the subject records may be impossible using current medical record storage and management device technologies. Therefore, there is a further need for medical record storage and management device tools that will allow third party access to patient medical records in the case of patients who are rendered unable to access the subject records, and/or to provide security access input on their own. A related need exists for tools that will allow patients to summon emergency assistance in the field, whereby upon arrival of the emergency provider the provider can access critical patient medical data that is accurate and securely stored and maintained with the patient. There is also a need for patients to be able to summon medical assistance quickly, optionally with the same device that stores and manages patient medical information. Summary of Exemplary Embodiments of the Invention.

To solve these problems and others, DNA medical ID bracelet provides a medical record storage and management device to which access is controlled by one or more levels of biometric authentication, alone or in combination with other methods of authentication and systems for creating, using and updating such a device. DNA medical ID bracelet further provides means for overriding the biometric authentication to all of the vital records in emergency situations and a means for summoning assistance if necessary.

DNA medical ID bracelet includes all of a patient's past and current medical records, past and present medications including dosage and duration, the patient's blood type, allergies, if any, and any treatment plan currently in place for a patient. The bracelet also includes documents such as advance medical directives, emergency contact information, all visited doctors and their contact information, past and present lab works and imaging information with complete radiology reports. DNA medical ID bracelet also has a pulse monitor and a GPS tracking system along with an emergency call button, “Amber” alert function with an LED digital screen that reminds the user what medication is scheduled for that day and when the next doctor or treatment appointment is scheduled.

A further benefit of the invention is that it is a wearable device that can be worn by the user. The data stored by the device may be static, in that it may not be altered, or fluid, in that the records can be updated each time a patient visits a healthcare provider or medical facility. In some embodiments, the DNA medical ID bracelet may be updated through a central server that compiles information from a variety of sources including health care providers.

There will not be any templates for patient-entered information; the information can only be entered by health care providers. The device allows a care provider or facility to upload information to the provider's system as well as record information to the bracelet. In some embodiments, new data is added sequentially to an existing record. In other embodiments, the device may integrate new material into existing records and/or generate current summaries based on new data received. The bracelet contains complete medical records that would be vital in an emergency situation.

In one embodiment, the DNA medical ID bracelet stores data from diagnoses that can be matched with patient symptoms, personal history, or family history. In other embodiments, upon receiving updated prescription information, a DNA medical ID bracelet can signal a warning of possible allergic reactions or negative drug interactions based on prior information and existing prescriptions stored in the device. In another embodiment, the device may transmit monitoring information to a care facility, for instance, when a patient is in an ambulance and being transported to the ER, anything that is being administered to the patient will relay in real time to the ER allowing the awaiting doctors to know exactly what is happening with the patient's current status and what needs to be done the moment the patient arrives. This process will allow the treating doctors to have complete knowledge of the patient's past and current condition and to be able to make informed and educated decisions on the patient's treatment process.

All of the information on the DNA medical ID bracelet will be encrypted or otherwise secured, requiring biometric authentication in order to unlock the bracelet and recover emergency contact information, blood type, current medications, drug allergies, past procedures if any, foreign objects inside the patient, insurance information etc..

There may be one or more levels of encryption or one or more levels of authorization or requirements for authentication for access to all medical records. In some embodiments, care providers may verify the ownership of the records on a DNA medical ID bracelet through a patient verification system. Such a system may include a comparison of a facial image of the patient along with a digital fingerprint identification process for extra security. In an extreme and dangerous situation security may be overridden by emergency services personnel. The encryption and other means for securing the data will meet FIPS (Federal Information Processing Standards) and HIPA (Health Insurance Portability and Accountability Act of 1996) standards.

The DNA medical ID bracelet will only be in the form of a (“USB”) or (Micro USB”) drives the device will be in the shape of a bracelet, for example, the device would be manufactured so that it could be worn, as watches or wrist bands, the reason for this design is the pulse monitor feature of the DNA medical ID bracelet, therefore, it has to be worn around the wrist to be able to detect the patient's pulse at all times.

Information transfer may occur using a USB connection, using Bluetooth, wirelessly or by any other means generally used to transfer data. In some embodiments, some information may be sent wirelessly while the transfer of other information may require a direct connection. Wireless transmission may occur by any means generally used to send information such as, but not limited to, radio signals, infrared signals, VHF signals, UHF signals, satellite signals, cellular signals, laser light, visible light, acoustic energy or radio wave signals.

The DNA medical ID bracelet also includes a GPS tracking capability system for sending a signal. This feature of the DNA medical ID bracelet works in conjunction with the DNA medical ID bracelet's pulse monitor. For example, in the event of a natural disaster such as an earthquake, hurricane, tornado or for our military personal, if the user is trapped under rubble or unconscious or if a soldier is separated from his/her unit, the pulse monitor will detect the user's pulse and the GPS tracking monitor will pinpoint their exact location for first responders or search and rescue teams to identify and recover the living first. The GPS tracking monitor also assists in cases of abductions or “AMBER” alerts in tracking and recovering the missing by pinpointing the user's exact location, dispatching law enforcement directly to the user's location. DNA medical ID bracelet includes an emergency function that includes a medical alert system. A medical alert system is a personal emergency response system designed to reduce complications associated with health-related emergencies, such as a slip and fall, where the user is unable to reach a phone or call for help. This function alerts the EMT's and first responders and a live operator comes online to aid and assist the user. If the user is unconscious or unresponsive, the GPS tracking system will pinpoint the users exact location and help will be dispatched to that location immediately. The emergency function may be activated by a single push button or other operation that initiates the medical alert system to contact emergency services. For example, activation of the emergency function initiating the medical alert system may send a signal to 911 or other emergency service numbers and the user's emergency contact person, or may contact the nearest hospital. For example, in the case of an elderly person, the single push button contacts 911, the person's spouse, the person's doctor and/or the assisted living facility where the person resides in any order chosen by the user or simultaneously.

Activation of the emergency function of the DNA medical ID bracelet may occur by any means generally used. A trigger may be mechanical, audio or electronic such as on a touch screen. Exemplary triggers include, but are not limited to, a panic button on the device, voice activation, a particular keying sequence, a touch screen button, a toggle, a slide, a strong impact sensor (e.g., for use in automatically transmitting a request in the event of a high-impact collision), a heat sensor (e.g., for use in automatically requesting emergency services in the event of a fire), a medical sensor (e.g., for automatically triggering a request for emergency services upon the detection of the cessation of the users pulse or other physical or medical condition).

In some embodiments, activation of the emergency function on the DNA medical ID bracelet may require a sequence of events, i.e. pushing a panic button one or more times. In another embodiment, the emergency function may be activated by holding a button on the DNA medical ID bracelet for a particular length of time (for example, 3, or 4 seconds).

When the medical alert system is initiated and a signal is sent, the signal may be sent by any means generally used to convey a signal to a third party. For example, the information may be sent by radio signal, infrared signal, a VHF signal, a UHF signal, a satellite signal, a cellular signal, laser light, visible light, acoustic energy, microwave signal, or a radio wave signal or other wireless means of conveying information. Once the medical alert system is activated, the system will also alert a designated family member or friend that may be nearby or in the same household.

Access to the information on the DNA medical ID bracelet may be granted by verification of one or more levels of biometrics. The stored medical and contact information will only be accessed via the DNA medical ID software.

Identification of the user of the DNA medical ID bracelet is secure by a digital fingerprint, and face recognition (a photo of the user) or a combination thereof. Verification of the user allows activation of the device for easy access by first responders, EMTs and emergency rooms. Different means of authentication or verification may be used to access information on the DNA medical ID bracelet. Verification of the user at one or more stages of accessing and or recording of information on the DNA medical ID bracelet may use the same or different means of authentication.

Authentication may be performed by the device itself, by inputting the USB bracelet into a computer hosting the DNA medical ID software. After authentication, the personal medical record may be transferred to a doctor or service provider's system either wirelessly or via Bluetooth by bumping their device against a compatible device running the same DNA software. In other embodiments, the personal medical records may be shared with any other healthcare provider via the secured DNA network with an online passcode or other encryption method including biometric identification of the person accessing a patient's medical records. In some embodiments, there may be an override to the biometric sensor, for example a code that can be entered by emergency personnel. Such a code may be carried on the bracelet itself, available to all hospitals or medical service providers, or by any other means generally available. In other embodiments, certain information may become accessible if an emergency signal is sent, such as a health care summary and/or list of current medications.

In some embodiments, the DNA medical ID bracelet can be updated from any computer or any other device that is equipped with the DNA software. For example, a care facility may gain access to a patient record through the Internet.

Embodiments herein describe a wearable Micro USB/USB bracelet containing vital updated medical records which can run applications capable of securely storing and managing medical records, and which is capable of assisting the user and conveying vital and accurate information to the relevant parties in an emergency situation. The systems and methods described herein may be contained in a single wearable bracelet or may be part of a network.

In some embodiments, the DNA medical ID bracelet includes a “panic” or medical alert button which will activate an emergency function and transmit a signal to a designated family member, emergency service responder, other care providers or coordinating individual or services. In some embodiments, the signal may contain emergency medical information regarding the owner of the DNA medical ID bracelet. In a preferred embodiment, the bracelet is capable of wirelessly transmitting an emergency alert signal activated by a subject wearing the bracelet. The emergency signal transmission may be actively or passively triggered as described herein. In these embodiments of the invention, the device is wearable and sized to be easily worn around the user's wrist.

In some embodiments, the DNA medical ID bracelet has a pulse monitor, as the bracelet is worn around the user's wrist it is constantly monitoring the user's pulse. In the event of an earthquake, hurricane, and tornado or for our men and women in the military, if the user is trapped under rubble or unconscious and unresponsive, the pulse monitor will detect the user's pulse and allow first responders or search and rescuers to locate and recover the living first. This function is also vital in an “Amber” alert or abduction situation. The casing of the DNA medical ID bracelet is fabricated from high-impact plastic or other durable material. In some embodiments, the casing may be impact resistant, tamper proof, electron shielded, electromagnetic pulse shielded or otherwise protected.

Medical Utility Hardware

DNA is an electronic identification apparatus in the form of a wearable bracelet. The DNA bracelet accompanies hardware such as a pulse monitor designed to detect pulse from the radial arteries. DNA includes an Emergency GPS Tracking System that can be utilized in the case of an emergency (i.e. natural disaster, medical emergency). The clasp of this bracelet is a micro USB/USB connector, which is sealed in an external cover. Depending on the product model, this micro USB/USB clasp can be opened conventionally, or unlocked with a fingerprint scanner. Other specialized models of DNA will have an alert system that contacts paramedics, and police (designed for the elderly, physically disabled, etc.). Data safeguarded within the DNA medical utility is unique to its user.

Product Description

DNA is a modern electronic identification apparatus that makes all data derived from medical procedures cohesive. Through the organization of information DNA helps form a digital relationship between:
Patient and Medical practitioner(s)
Medical Practitioner(s) and Medical Practitioner(s)

DNA encrypts, and secures all past, and present medical history into its smart archive. Only medical organizations, medical facilities, medical groups (i.e. Emergency Medical Technicians), and specific medical practitioners (i.e. doctors, medical specialists, surgeons, etc.) vetted by our organization will be able to input information into DNA. A patient's status can be updated to reflect their most noteworthy health condition(s), however medical history cannot be edited, or altered in any form; it remains archived as historical data. A new doctor will be able to better treat a patient when he/she reviews the information within the patient's DNA bracelet; that information is inputted by doctors who have already worked with the patient.

DNA is made to make data transfers consistent, and convenient. A systemic flow of information makes DNA accurate in the case of emergencies. Paramedics can update patient-health status in real-time; this allows for doctors to know what to expect from an incoming patient, and to make an informed decision on how to operate.

DNA's transparency is to create a seamless exchange of medical information to all parties involved. Through data collection, database, distribution, data visualization, and more the process is interlinked, and simplistic. The DNA bracelet, like deoxyribonucleic acid, is connected and unique to its user; within the DNA bracelet is DNA software virtualized to provide simplified and elaborated medical information to all medical practitioners dealing with the sole owner, and user of DNA. The first tab of our software acts as an electronic ID, and holds vital information on the user's most pertinent medical data (i.e. allergies, medical conditions, blood type, past/present prescriptions, etc.); this page is made to verify the user, and to minimize the chance of medical malpractice in an emergency.

### EXAMPLES

For the first time ever DNA will put all doctors on the same page.

DNA will provide instant, vital medical information to first-responders and emergency rooms (in an emergency, every second can be the difference between life and death).

DNA's pulse monitor and Emergency GPS Tracking System will aid first responders in case of natural disasters allowing them to rescue the living first. The Emergency GPS Tracking System also has military applicability; this feature will allow for medics to locate fallen soldiers and for personnel to retrieve lost servicemen.

DNA will help prevent 450,000 annual deaths that occur from taking medication exactly prescribed by your doctor(s) (helps prevent lethal mix of medications).

DNA will help prevent medical malpractice and doctor's mistakes.

DNA could possibly save insurance companies billions of dollars by preventing medical malpractice and accidental deaths; casualties. Insurance companies will have to compensate.

### REFERENCES

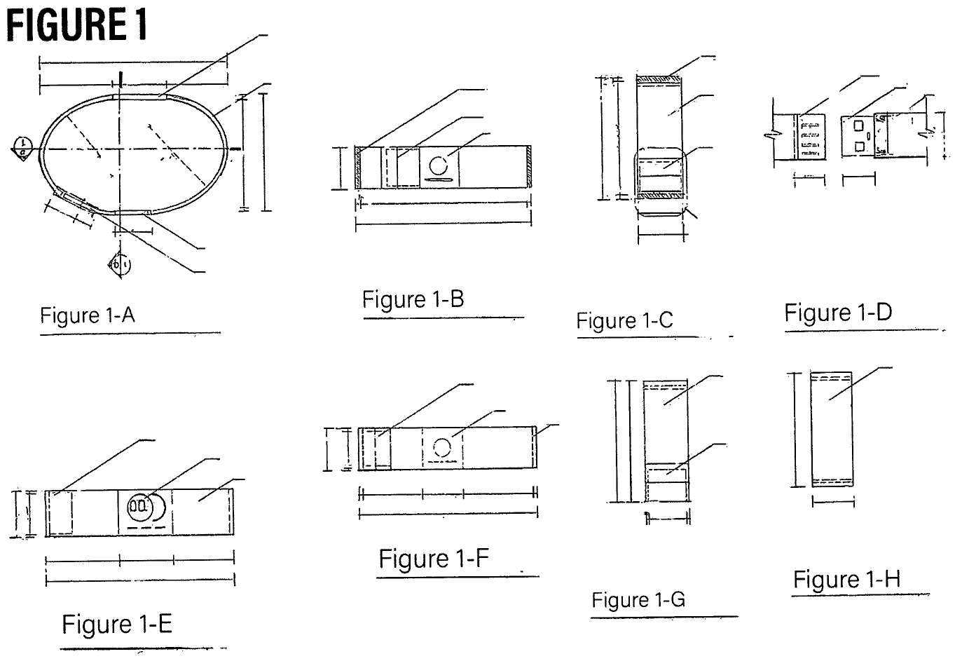

FIG. 1—Drawing

FIG. 1-A: Overview of the DNA bracelet.

FIG. 1-B: Panic button

FIG. 1-C: LCD screen

FIG. 1-D: USB clasp (opened)

FIG. 1-E: USB clasp (closed)

FIG. 1-F: Pulse monitor

FIG. 1-G: GPS

FIG. 1-H: Impact sensor

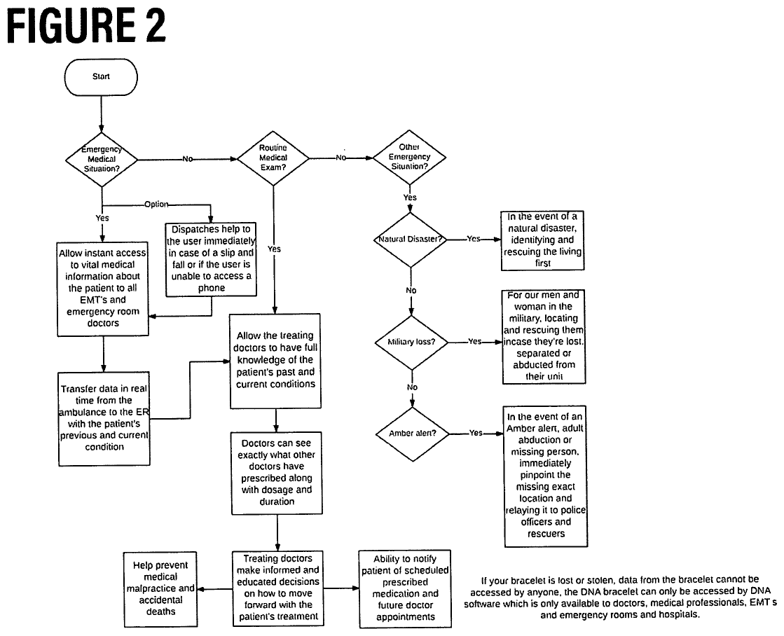

FIG. 2—Flow Chart

We claim:

1. A wearable USB/Micro USB medical record storage and management device comprised of:
A mass past and present medical records storage device;
A high impact sensor for falls and car accidents;
A pulse monitor;
A wireless GPS system;
An emergency EMS/FIRE/POLICE button
An emergency AMBER alert button;
A microphone and speaker for live operator assistance;
A doctor's appointment display reminder and lethal medication combination warning display;
Wherein the medical record storage and management device is wearable.

2. The device of claim 1, wherein the mass storage device is a USB/Micro USB bracelet.

3. The device of claim 1, wherein the medical record storage and management device is part of a wearable bracelet.

4. The device of claim 2, wherein the USB flash drive weighs between 20 g and 50 g.

5. The device of claim 2, wherein the USB flash drive is between 1 inch and 3 inches long.

6. The device of claim 1, wherein the mass storage device is a USB/Micro USB drive.

7. The device of claim 1, wherein the medical record storage and management device is a USB/Micro USB drive.

8. The device of claim 1, further comprising a pre-recorded message.

9. The device of claim 8, wherein the pre-recorded comprises identification information, past and current up-to-date medical history.

10. The device of claim 1, wherein the high impact sensor is capable of alerting EMS, FIRE, POLICE and designated family members.

11. The device of claim 1, further comprising a pulse monitoring.

12. The device of claim 1, wherein the pulse monitor system detects the users pulse.

13. The device of claim 1, further comprising a global positioning system.

14. The device of claim 1, further comprising a locator system.

15. The device of claim 14, wherein the locator system is a GPS locator.

16. The device of claim 1, wherein the wireless transmitter is capable of sending a radio signal, an infrared signal, a VHF signal, a UHF signal, a satellite signal, laser light, visible light, acoustic energy, a cellular signal, or radio wave signal.

17. The device of claim 1, further comprising an emergency signal button.

18. The device of claim 17, wherein the wireless transmitter is capable of sending a signal when an emergency button is depressed.

19. The device of claim 1, wherein the wireless transmitter is capable of sending a signal when a strong impact is detected, if a heat sensor reaches a threshold temperature, or upon detection of a change in the physical state of the wearer.

20. The device of claim 18, wherein the signal sent by the wireless transmitter is sent to an emergency services provider.

21. The device of claim 18, wherein the signal sent by the wireless transmitter is sent to a monitoring service.

22. The device of claim 1, wherein the device further comprises an AMBER alert button.

23. The device of claim 22, wherein the AMBER alert button notifies the authorities of the users exact location.

24. A method of securely storing and managing up to date medical records in a wearable device comprising:
providing a medical record storage device to be worn by a patient; authenticating the holder of the device; and transferring medical records to the device; wherein the transferred medical records can integrate into medical records already on the device.

25. The method of claim 24, wherein authenticating the holder of the device comprises using face recognition or touch ID.

26. The method of claim 25, wherein the biometric sensor is capable of fingerprint recognition, or face recognition.

27. The method of claim 24, further comprising allowing access to the medical records by emergency personnel.

28. A method of using securely stored medical records in a wearable device comprising: providing a medical record storage device to be worn by a patient; authenticating the identity of the patient whose medical records are on the device;
securely accessing the records on the device; accessing monitoring information in the device;
and treating the patient based on the monitoring and medical record information contained in the device.

29. The method of claim 28, wherein the identity of the patient is authenticated using touch ID of facial recognition.

30. The method of claim 29, wherein the biometric sensor is capable of fingerprint recognition, or face recognition.

31. The method of claim 28, wherein the monitoring information is transmitted to a care facility and treatment instructions are transmitted back to the portable device.

32. A system for providing medical records to a wearable device, the system comprising:
An interface that receives input from the mobile device;
A biometric sensor for verifying the user of the mobile device;
An automated information gatherer that in response to the received input compiles records from the user and medical service providers;
A means for reformatting the obtained records;
An interface that displays the compiled records on the wearable device.

33. The system of claim 32, wherein the mobile device is a wearable USB/Micro USB device that receives content from the Internet.

34. The system of claim 32, wherein the system further comprises a means for locating the device.

35. The system of claim 32, wherein the means for locating the wearable mobile device comprises the use of a global positioning system.

36. The system of claim 32, wherein the system further comprises a means for contacting an emergency services provider.

37. A wearable mobile device capable of running an operating system, wherein the mobile device comprises: one or more files containing electronic medical records;
A biometric sensor;
A means for contacting another device;
A means for displaying or communicating a pre-recorded message; and a locating system.

38. The mobile device of claim 37, wherein the wearable device is a bracelet a wireless-enabled personal digital assistant that receives content from the Internet.

39. The mobile device of claim 37, wherein the biometric sensor is capable of voice recognition, handprint recognition, face recognition and touch ID.

40. The wearable device of claim 37, wherein the medical records are stored in a secure format.

41. The wearable device of claim 37, wherein the locating system is a global positioning system (GPS).

42. The wearable device of claim 37, wherein the wearable device can transmit its location to another device.

43. The wearable device of claim 37, wherein the other device is a device used by Emergency services personnel.

44. The wearable device of claim 37, wherein the pre-recorded message is an audio message.

45. The wearable device of claim 37, wherein the pre-recorded message is a visual message.

46. The wearable device of claim 37, wherein the pre-recorded message contains emergency medical information regarding a user of the bracelet.

47. A method for securely storing medical and managing medical records adapted for use in a wearable bracelet USB/Micro USB device comprising: authenticating a user biometrically;
displaying a summary of medical information;
allowing ONLY a doctor or health care facility to update medical information;
adding the medical information to a category of medical information; and updating the contents of the wearable device to include the new information.

48. The method of claim 47, wherein the biometric authentication is voice recognition, handprint recognition, or face recognition.

49. The method of claim 48, wherein the medical records are updated onto the bracelet from a computer.

50. The method of claim 50, wherein the contents of the wearable device are updated via the Internet.

51. A medical record storage and management application for use in a wearable bracelet having an operating system capable of executing the application comprising: one or more files containing electronic medical records stored in a secure format;
A biometric authorization protocol;
A means for updating the electronic medical records;
A means for viewing the electronic medical records.

52. The medical record storage and management application of claim 52, wherein the application further comprises a protocol for contacting an emergency services provider.

53. The medical record storage and management application of claim 53, wherein the application further comprises a pre-recorded file containing personal identification and medical information regarding the user of the bracelet.

54. The medical record storage and management application of claim 54, wherein the pre-recorded file is accessible by emergency services personnel.

55. The medical record storage and management application of claim 54, wherein the application further conveys location information to emergency services personnel.

56. The medical record storage and management application of claim 54, wherein. The location information is obtained using a global positioning system.

57. The medical record storage and management application of claim 54, wherein the location information is obtained using cell tower triangulation.
